(19) United States
(12) Patent Application Publication
(10) Pub. No.: US 2010/0315589 A1
(43) Pub. Date: Dec. 16, 2010

(54) TORIC OPHTHALMIC LENS

(76) Inventor: VALDEMAR PORTNEY, Newport Coast, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 12/485,228

(22) Filed: Jun. 16, 2009

(51) Int. Cl. G02C 7/04; A61F 2/16

(52) U.S. Cl. 351/160 R; 623/6.23; 623/6.24

(57) ABSTRACT

An ophthalmic lens for placement on the human eye or implanted into an eye is described. The lens has a cylinder power for eye astigmatism refraction error correction and incorporates aspherization of at least one of the surfaces to reduce vision quality reduction with toric ophthalmic lens rotation that creates meridional misalignment as compared with the equivalent toric lens with non-aspherized surface.

### FIELD OF THE INVENTION

The present invention relates to toric ophthalmic lenses which provide cylinder power to correct eye astigmatism refraction errors.

### BACKGROUND OF THE INVENTION

Astigmatism is meridian-dependent refraction error of an eye. This is usually due to toroidal shape of at least one of the ocular surfaces of the eye, most commonly the anterior corneal surface. This type of astigmatism is called corneal astigmatism. Toroidal shape is two-curvature shape described by a surface with meridians of steepest and flattest curvatures located at right angle to each other. This is also called regular astigmatism which is correctable by an optical aid such as spectacles, contact lens, corneal implant or intraocular lens.

Astigmatism may also be due to an ocular surface of the eye is transversely displaced or tilted, most commonly a surface of crystalline lens in phakic subjects. This type of astigmatism is called lenticular astigmatism. Lenticular astigmatism almost invariably manifests the flattest meridian close to vertical orientation and usually does not exceed 1.5 D cylinder. Corneal astigmatism on the other hand, manifests large variation in the meridian orientation and magnitude.

Astigmatism can be corrected by a toric contact lens or toric intraocular lens. Later can be phakic or aphakic intraocular lens, i.e. implanted in a subject with the natural lens either intact or removed. The astigmatism correction requires proper toric lens orientation—the lens flattest meridian to be aligned with the steepest meridian of the cornea, or steepest meridian of the eye refraction error if lenticular astigmatism is considered.

Astigmatism correction commonly involves correction for other ocular deficiencies such as myopia (nearsightedness), hyperopia (farsightedness), aphakic and presbyopia, and the corrective toric ophthalmic lens may include spherical corrective power and multifocal corrective power for the corresponding ocular deficiency correction.

The toric lens alignment in reference to ocular astigmatism meridian, so called meridional alignment, is the most critical factor of a toric lens performance requirement in order to allow maintaining an acceptable image quality.

The attempt to reduce sensitivity to toric lens meridional misalignment can be found in U.S. Pat. No. 5,570,143 by Newman where aspheric surface shape that induced a depth of focus was discussed. The lens according to Newman's invention includes optical topography on the surfaces of the lens which induces a depth of focus. When a lens meridians line up with the meridians the cylinder power requirement is fulfilled. If the meridians do not line up, i.e. a condition of meridional misalignment, the author speculates that the depth of focus corrects for the resulted overrefracton. The Newman's patent references to 1.5 D depth of focus as an example. The fundamental issue with such consideration is that it relies only on the refraction power consideration without taking into account resulted image quality. For instance, an asphericity that significantly impacts the depth of focus must be at least 1 D range in order to be clinically significant and such level of asphericity reduces the image quality even before considering meridional misalignment. A meridional misalignment of the toric lens resulted in residual astigmatism and spherical aberration that further reduce the image quality. Based upon the refraction stand point, the depth of focus blends the corresponding residual refraction error but the Newman's patent is silent about the resulted image quality. In addition, no disclosure of the particulars of the aspheric surface that increases depth of focus in toric lens was provided.

Another attempt to address meridional misalignment can be found in U.S. Pat. No. 5,796,462 by Roffman. The Roffman's patent describes asphericity application to each toric meridian in a form of a prolate aspheric curve. Similar to Newman's disclosure, the patent relies on the increased depth of focus to reduce sensitivity to the meridional misalignment but also applying it to the toric surface in such a way that the effective cylinder decreases from the center of the lens towards the periphery of the optical zone. The issue with this approach is that the described aspherization leaves residual cylinder even with lens perfect meridional alignment. In addition, no disclosure of the particulars of the aspheric surface of the lens was provided except the reference to the prolate shape of surface aspherization.

Additional difficulty with the above prior arts is that the aspherization that increases depth of focus must be effective within about 3 mm pupil which is an average pupil size at normal photopic light condition. The disclosures are silent about an impact of an aspherization at larger pupil where the depth of focus might be even broader than at 3 mm pupil leading to image degradation.

In view of the prior arts limitations, there is a need for improvement of the toric ophthalmic lens design that reduces sensitivity to meridional misalignment.

### SUMMARY OF THE INVENTION

A lens in accordance with the present invention consists of front (anterior) and back (posterior) optical surfaces.

A toric shape formed into one of the anterior and posterior surfaces with the toric shape being defined by undulating curvatures along meridians of the toric shape. At least one of the curvatures produces different signs of longitudinal ray aberration within about 3 mm pupil diameter.

A toric lens may consist of a single optical element or multiple optical elements. At least one of the optical elements comprises of an anterior surface having an anterior optical zone, and an opposite posterior surface having posterior optical zone, wherein one of the anterior optical zone or the posterior optical zone is a toroidal surface whereas the other zone is non-toroidal surface which is a spherical surface or an aspheric surface according to the present invention. A toroidal surface may also be a toroidal spherical or a toroidal aspheric according to the present invention. The toroidal and non-toroidal surfaces in combination provide a targeted cylindrical refraction power and a targeted spherical refraction power for distance vision. The toric ophthalmic lens of the present invention may also comprise of a non-toroidal multifocal or a toroidal multifocal surface of the bifocal type, i.e. the surface that is designed to produce two distinct foci for far and near vision. The bifocal surfacer of such design may be of diffractive optic type that produces far and near foci by utilizing appropriate diffraction orders or refractive optic type that incorporates the zones of two distinct powers, one for far and another for near vision. The specificity of the bifocal design is that it doesn't incorporate a clinically significant continuous foci range of at least 1 D.

Image at the retina directly depends upon transverse ray aberrations. Due to the more complex description of the transverse ray aberration which involves 2-dimensional characterization, it is more illustrative to describe the invention in terms of a longitudinal ray aberration graph which involves 1-dimensional characterization. A Longitudinal Spherical Aberration graph is a graph depicting optical ray intersections at the optical axis as the rays passing at different distance from the lens optical center. The lens is considered in centered position in reference to the optical axis and the point-source of the optical rays is considered at infinity. Optical design program such as Zemax® Optical Software incorporates graphical representation of longitudinal spherical aberration (LSA) and is used for the present invention illustration where the horizontal axis of the graphical representation represents optical axis with zero point coordinate defines the focus position and vertical axis represents distance from the aperture center which coincides with the position of the optical axis.

If longitudinal spherical aberration changes in one direction, increases or reduces, with the distance from the lens optical center, one can called the corresponding LSA a prolate type and corresponding aspherical surface also prolate type as it is similar to that of the LSA produces by a conic aspheric surface. One can call LSA a non-prolate type if the direction of its plot changes, i.e. LSA graph plots towards the lens and then changes direction in farther away from the lens or visa versa, farther away from the lens and then towards the lens. The responsible for the shape aspheric surface can also be called non-prolate aspheric surface. The LSA plot direction can be represented by LSA sign—“minus” sign for the LSA plot changes towards the lens and “plus” sign for the LSA plot changes farther away from the lens.

The present invention involves a longitudinal ray aberration with different signs within about 3 mm pupil which is an average pupil size at normal, so called photopic light condition. In addition, the range of LSA from the closest point to the furthest point from the lens within 3 mm aperture is usually below a range of foci distribution applicable in multifocal optic with the continuous range of foci, i.e. the range utilized in the present invention does not exceed 1 D range in dioptric power term thus maintaining primarily monofocal lens characteristic. The present invention accepts that the dioptric power range may reach beyond 1 D range but only within insignificant area of less that 10% of 3 mm aperture.

Examples of image quality produced by the prior art and the image quality produced by the present inventions are interpreted in terms of Modulation Transfer Function (MTF). The MTFs have been compared at the condition with perfectly aligned meridians and with meridional misalignment of different degrees.

The present invention is applicable to intraocular lens including phakic and aphakic lenses with single and multiple optical elements, as well as to contact lenses and corneal implants. It is also applicable to bifocal optic which is designed to produce only far and near foci.

The unexpected outcome of the present invention is that if a toric ophthalmic optic produces longitudinal ray aberrations of different signs within 3 mm aperture i.e. non-prolate type, and the area associated with each sign is significant enough, i.e. at least 10% if 3 mm aperture, the resulted aspherization of the toric lens preserves image quality with meridional misalignment to larger degree than the equivalent toric lens without the corresponding asphenrzation.

### BRIEF DESCRIPTION OF THE FIGURES

FIG. 1 illustrates a toric surface configuration.

FIG. 2 illustrates an example of longitudinal spherical aberration (LSA) per prior art that extends depth of focus as disclosed by Newman

FIG. 3 demonstrates the image quality in terms of Modulation Transfer Function of the eye with the toric lens aspherization that increases depth of focus which corresponds to the LSA on FIG. 2. The MTF is compared with the toric spherical MTF at the same meridional misalignment.

FIG. 4 illustrates an example of longitudinal spherical aberration per the prior art that extends depth of focus and reduces cylinder magnitude towards optical zone periphery as disclosed by Roffman.

FIG. 5A and 5B provide the image quality in terms of Modulation Transfer Function of the eye with the toric lens aspherization that increases depth of focus and reduces cylinder magnitude towards optical zone periphery which corresponds to the LSA shown on FIG. 4. The MTF is compared with the toric spherical MTF at the meridional alignment and meridional misalignment.

FIG. 6 demonstrates an example of aspherized per the present invention the toric surface shape along Y- and X-meridians of steepest and flattest curvatures produced by toric intra-ocular lens.

FIG. 7 demonstrates the example of longitudinal spherical aberrations plots per the present invention particularly one produced by the surface aspherization shown on FIG. 6 and spherical longitudinal aberration plot of the equivalent toric lens with non-aspherized surface.

FIG. 8A, 8B and 8C are the examples of Modulation Transfer Functions (MTFs) of the eye with toric aspherical ophthalmic lens according to the present invention per toric aspheric surface shown on FIG. 7. The MTFs are compared with the MTFs of the equivalent toric non-aspherized lens per the present invention. The MTFs are in meridional alignment and different meridional misalignments.

### DETAILED DESCRIPTION

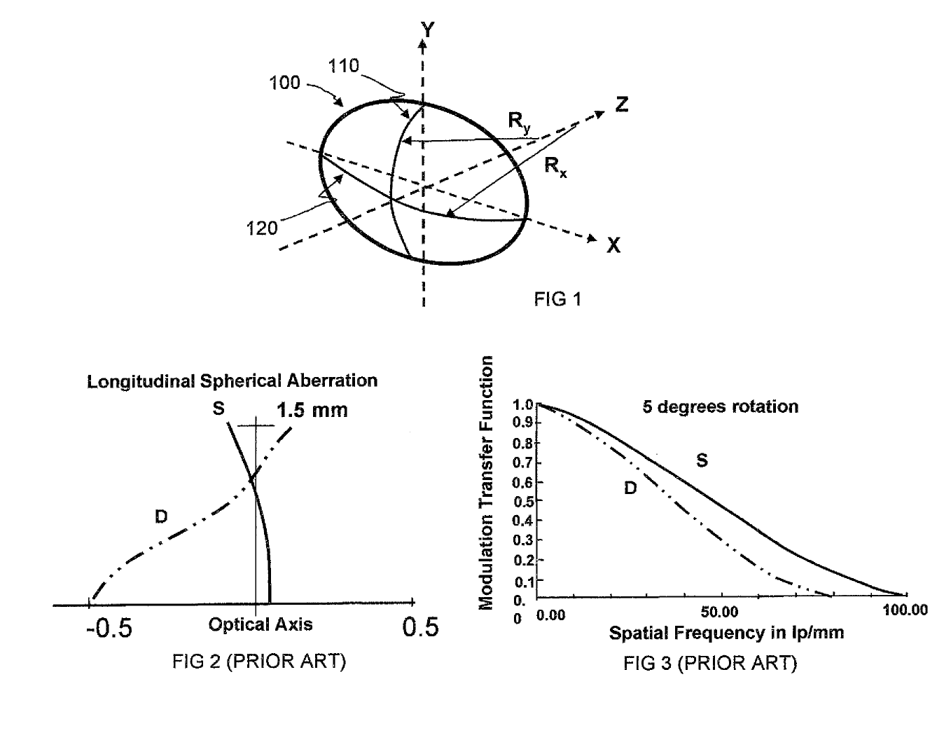

FIG. 1 illustrates a toric surface configuration suitable for use as a contact lens, intraocular lens, single element lens or a multiple element lens. The optical axis coincides with Z-axis and the shortest radius R_{y} (steepest curvature) is along y-meridian which is at the same plane as Y-axis and the longest radius Rₓ (flattest curvature) is along x-meridian which is at the same plane as X-axis. The toric surface 100 is characterized by the meridians along the steepest curvature, 110, and flattest curvature, 120. It could be that the flattest curvature is along y-meridian and steepest curvature is along x-meridian. For illustrating purposes, this type of meridional orientation in reference to Y- and X-axis has been chosen. In a spheric toric lens, R_{y} and Rₓ are constant and the in an aspheric toric lens R_{y} and Rₓ are variable, that is, the toric surface has an aspheric cross section at the major and minor axes x-y.

Toric surface Cylinder is defined by the difference in dioptric power between the meridians. The toric surface Spherical power is defined by the average dioptric power between the meridians or the dioptric power along one of the meridians depending upon the choice of toric lens refraction designation.

FIG. 2 illustrates an example of longitudinal spherical aberration per the prior art that extends depth of focus. The Graph S represents LSA of the equivalent toric spherical lens along one of the meridians and Graph D represents the LSA of the lens along equivalent meridian with extended depth of focus of slightly more than 1 D in order to model a minimally clinically significant depth of focus increase over the equivalent spherical lens of the same power. The comparison is for 3 mm pupil. The shape of the LSA Graph D is determine by the optical consideration that a contribution to depth of focus reduces toward the lens center and, as a result, the LSA range increases towards the lens center to take this into account.

FIG. 3 provides the image quality in terms of Modulation Transfer Function of the eye with the toric lens aspherization that increases depth of focus per the corresponding LSA Graph D of the FIG. 2 at the same toric meridian. The cylinder power of the corresponding lens was 2.5 D at spectacle plane. The MTF is compared with the MTF produced by the conventional toric spherical lens of the same spherical and cylinder powers and both at the meridional misalignment of 5 degrees. The MTFs where taken at the best focus position defined by the highest MTF at the condition of meridional alignment where astigmatism refraction error is fully corrected because this is the condition for which the toric lens is prescribed.

The result demonstrates that the MTF of the toric lens with increased depth of focus actually may be lower the MTF of the equivalent spherical lens at the meridional misalignment. The FIG. 3 demonstrates the limitation of the design that is solidly based upon refraction consideration of the prior art without taking into account the image quality—range of powers associated with the depth of focus increase corrects for the overrefracton resulted from the meridional misalignment but the image quality may fall below the image quality of the equivalent toric lens designed that doesn't incorporate depth of focus increase.

The optical analysis and clinical outcomes indicate that the design of ophthalmic lens with the increased depth of focus has a benefit in reducing sensitivity to eye astigmatism refraction error if no toric topography is added as compared with corresponding spherical lens which also doesn't include a toric topography. In this case, the overrefraction resulted from the presence of the astigmatism refractive error is partially compensated by the presence of depth of focus.

If toric topography is added to the lens designed with increased depth of focus, the effect on image quality reduction may be excessive—the sensitivity to meridional misalignment may be actually increased as compared with the corresponding toric ophthalmic lens without depth of focus increase. The explanation of this outcome is that a clinically significant depth of focus of more than 1 D reduces image quality of the optic to the level that the additional aberrations resulted from the meridional misalignment of the tonic lens significantly impact the image quality and reduces it below the level manifested by the corresponding lens without the depth of focus increase.

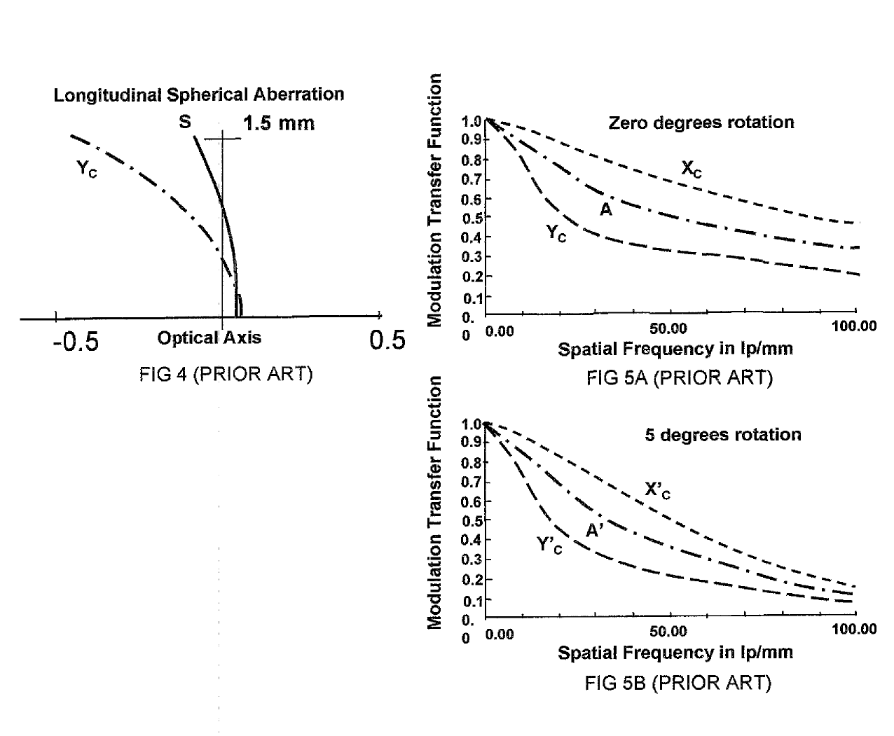

FIG. 4 illustrates an example of longitudinal spherical aberration, Graph Y_{c}, per the prior art that extends depth of focus and reduces cylinder magnitude towards optical zone periphery. The Graph Y_{c} is shown in comparison with Graph S of the toric non-aspherized lens along the same y-meridian. The lens of this example is with 2.5 D cylinder at the spectacle plane and y-meridian of the toric aspherized lens is the flattest curvature meridian. The aspherization along y-meridian is produced by conic curve or prolate aspheric as dictated by the corresponding prior art, and the plot of LSA Graph Y_{c} is shown as changing towards the lens (left) with distance from the optical center, i.e. the instance power increases along y-meridian towards the power magnitude of the steepest curvature at x-meridian. Thus, the difference in dioptric powers between the meridians, i.e. effective cylinder, decreases from the center of the lens towards the optical zone periphery within 3 mm aperture.

FIG. 5A and 5B provide the image quality in terms of Modulation Transfer Function of the eye with the toric lens aspherization that increases depth of focus and reduces cylinder magnitude towards optical zone periphery along y-meridian as shown on FIG. 4. The MTFs were taken at the best focus defined by the highest average MTF at the condition of meridional alignment.

The outcome is an incomplete astigmatism correction even with meridional alignment as demonstrated by significant difference in MTF magnitudes between y-meridian, Y_{c} plot and x-meridian, X_{c} plot, FIG. 5A. The difference in MTF magnitudes in different meridians may cause vision issues analogous to the astigmatism such as image distortion. The difference in MTF magnitudes between different meridians is largely preserved with meridional misalignment of 5 degrees as shown on FIG. 5B, Y′_{c} plot of the MTF in y-meridian and X′_{c} plot of the MTF in x-meridian. The FIGS. 5A and 5B also include the Average MTFs at both conditions of meridional misalignment, A plot and A′ plot. The Average MTF indicates that the toric lens designed with the cylinder magnitude reduction towards lens periphery may indeed achieve higher resolution as comparing with non-aspherized toric lens MTF shown by S plot on FIG. 3 and claiming by the corresponding prior art the MTF magnitude of Graph A′ of FIG. 5B at high spatial frequencies is higher the MTF magnitude of the non-aspherized toric shown by Graph S of FIG. 3, but the average MTF improvement is at the expense of the image distortion due to the substantial differences in the MTFs at different meridians.

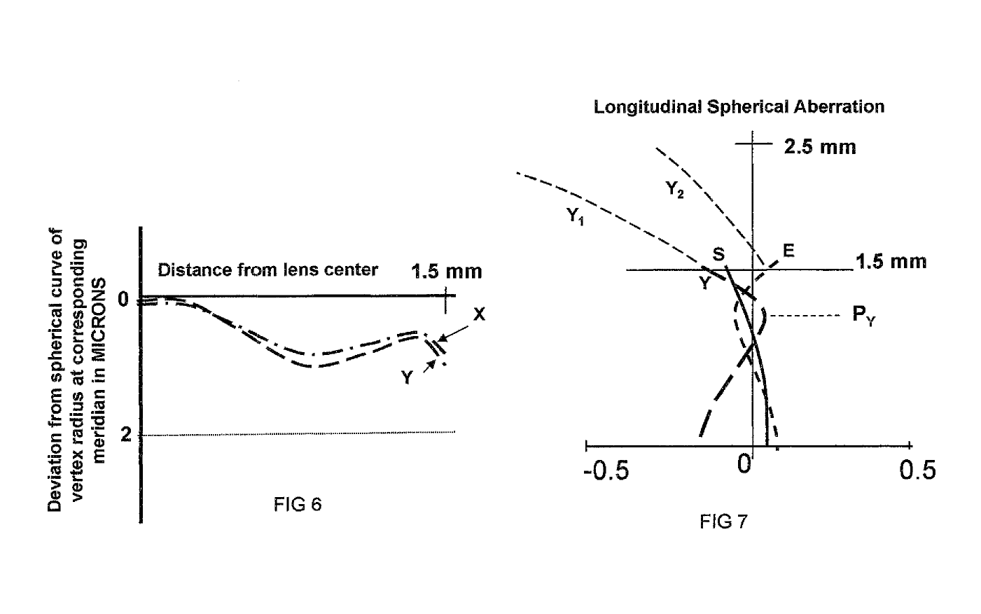

FIG. 6 demonstrates an example of aspherized per the present invention toric surface shape along y- and x-meridians of flattest and steepest curvatures correspondently that produces 2.5 D cylinder at spectacle plane. Toric intra-ocular tons was used in this example. The curve characteristics along y-meridian are designated by “y subscript” and along x-meridians are designated by “x subscript” and both are plotted as the deviations from the corresponding spherical shapes defined by the vertex radii in y- and x-meridians.

Aspheric toric surface of the toric lens can be described by the following equations along y- and x-meridians:

$$z ( y ) = \frac{{c}_{y} {y}^{2}}{1 + \sqrt{( 1 - {c}_{y}^{2} {y}^{2} )}} + {A}_{y 2} {y}^{2} + {A}_{y 4} {y}^{4} + {A}_{y 6} {y}^{6} + …$$

$$z ( x ) = \frac{{c}_{x} {x}^{2}}{1 + \sqrt{( 1 - {c}_{x}^{2} {x}^{2} )}} + {A}_{x 2} {x}^{2} + {A}_{x 4} {x}^{4} + {A}_{x 6} {x}^{6} + …$$

where “y” and “x” is the distance from the lens center along y- and x-meridians correspondently, “c_{y}” and “cₓ” are vertex curvatures along y- and x-meridians correspondently, c_{y}=1/R_{y} and cₓ=1/Rₓ with R_{y} and Rₓ being vertex radii along y- and x-meridians correspondently. Coefficients A_{y2}, A_{y4}, etc are aspheric coefficients along y-meridian and Aₓ₂, Aₓ₄, etc are aspheric coefficients along x-meridian.

Table 1 below lists the example of specifications of toric intraocular lens with toric aspheric surface according to the present invention.

TABLE 1
Toric Aspheric IOL with 2.5 D Cylinder at spectacle plane, n = 1.489

| Parameters | y-meridian | x-meridian |
|---|---|---|
| Front spherical radius | 9.55 | 9.55 |
| R (mm) | | |
| Back toric meridional | −30(*) | −17.616(*) |
| vertex radii R_{y} and Rₓ | | |
| (mm)) | | |
| A_{y2} and Aₓ₂ | −0.002825423 | −0.0022403627 |
| A_{y4} and Aₓ₄ | 0.0026735707 | 0.001905198 |
| A_{y6} and Aₓ₆ | −0.000720278 | −0.00048008386 |

(*)negative radius value for posterior convex surface

The aspheric shape along a meridian demonstrates wavy or undulating nature of the deviation from the spherical shape defined by the vertex radius along the corresponding meridian. This reveals a variable nature of surface curvature that increases and decreases in its magnitude from surface center toward surface periphery within 3 mm diameter. This is the demonstration of non-prolate surface aspherization as compared with prolate surface aspherization where the surface curvature either continually increases or reduces from the center towards the surface periphery.

A similar aspherization can be also applied to the opposite non-toric surface of the toric lens by similar equation shown below:

$$z ( r ) = \frac{{cr}^{2}}{1 + \sqrt{( 1 - {c}^{2} {r}^{2} )}} + {A}_{r 2} {r}^{2} + {A}_{r 4} {r}^{4} + {A}_{r 6} {r}^{6} + …$$

where “r” is the distance from the lens center, “c” is vertex curvature, c=1/R with R being vertex radius, and Aᵣ₂, Aᵣ₄, etc are aspheric coefficients.

FIG. 7 demonstrates the longitudinal aberrations plot per this invention resulted from the non-prolate surface aspherization per FIG. 6, Graph Y. The FIG. 7 also includes the LSA plot produced by the eye with the equivalent toric spherical lens along the same y-meridian, Graph S. The Graph Y demonstrates non-prolate nature of the corresponding aspherization, the LSA demonstrates positive sign up to the distance P_{y} from the center and then negative sign towards 1.5 mm distance from the center. The design incorporates substantial enough regions of different signs of at least 10% of the area each within 3 nmn aperture. Beyond 1.5 mm distance from the center, the LSA plot may be a continuation of the Graph Y, Graph Y₁, or shifted along the optical axis, Graph Y₂. The peripheral portion of the toric lens surface may be also aspherized for contrast sensitivity improvement at large pupils above about 4 mm diameter usually manifested in low light or scotopic conditions.

An aspherization according to the present invention is also possible with the LSA producing negative sign at the region closer to the center and then negative sign at the peripheral region towards 1.5 mm from the lens center, Graph E.

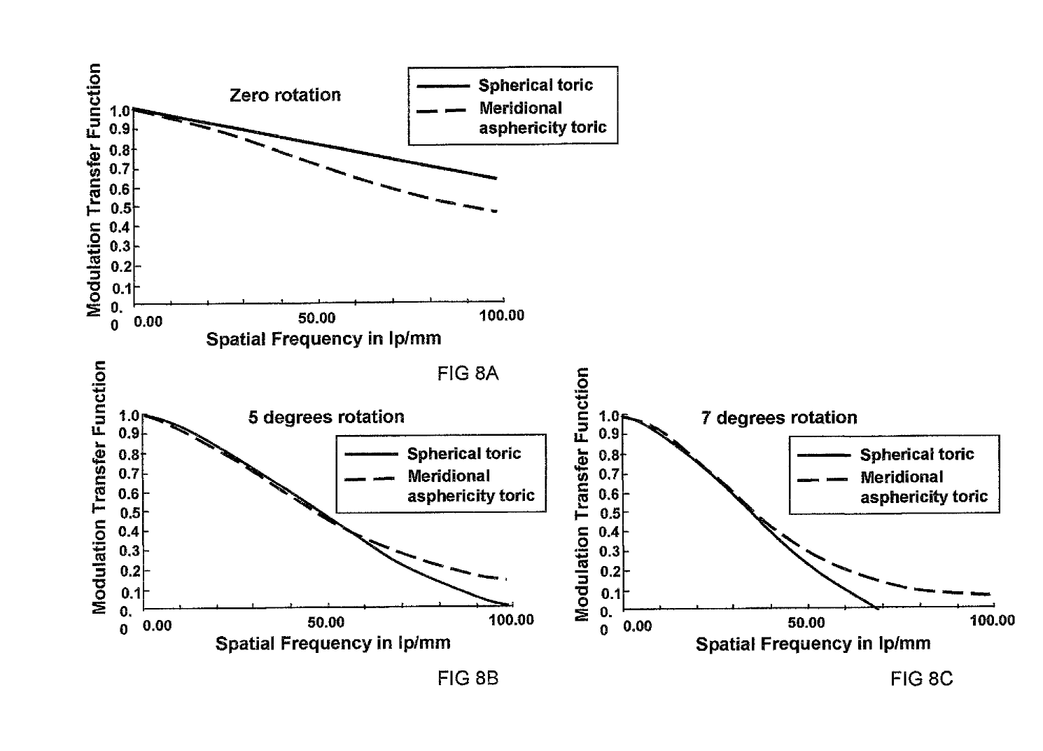

FIG. 8A, 8B and 8C demonstrate the MTFs of the eye at 3 mm aperture with toric aspherical lens per the present invention as described on FIGS. 6 and 7 and specified by the Table 1. The corresponding MTF of non-prolate toric is compared with the MTF of the equivalent toric spherical lens, both at different degrees of meridional misalignments—at zero misalignment, 5 degrees and 7 degrees. The MTFs were taken at the best focus defined by the highest MTF at the condition of meridional alignment where the astigmatism refraction error is fully corrected and no residual astigmatism is present. The MTFs of the aspherical toric lens are plotted by broken lines and the MTFs of non-aspherical lens, i.e. toric spherical lens, are plotted by solid lines.

At zero misalignment, the aspherical MTF is lower the corresponding non-aspherical MTF because the aspherization introduces additional spherical aberration. The MTF is still fairly high and meets ISO 11979-7:2006 standard requirement for monofocal IOLs of minimum of 0.28 MTF level at 100 lp/mm of spatial frequency. With meridional misalignment of 5 degrees, the toric spherical MTF drops to the limit of resolution at 100 lp/mm which corresponds to 20/20 Vision Acuity. The MTF of the aspherized toric lens at the same misalignment demonstrates similar magnitudes for up to about 60 lp/mm spatial frequency but then the MTF level is substantially higher for higher spatial frequencies. With meridional misalignment of 7 degrees, the toric spherical MTF drops to the limit of resolution slightly below 60 lp/mm of about 20/35 Vision Acuity. The MTF of the aspherical toric lens at the same misalignment demonstrates the same magnitudes for up to 40 lp/mm of spatial frequency but the MTF is substantially higher for higher spatial frequencies reaching above zero level at 100 lp/mm thus still demonstrating 20/20 Visual Acuity.

The toric lens aspherization per present invention demonstrates that the aspherical toric lens may provide 20/20 Visual Acuity even at 7 degrees meridional misalignment but the equivalent toric spherical lens can only reach 20/35 Visual Acuity which more than two lines reduction in visual acuity and fairly close to the limit of the acceptable ophthalmic lens performance of 20/40 Visual Acuity.

What is claimed is:

1. An ophthalmic toric lens to be worn on an eye or implanted inside of an eye, the lens comprising:
an anterior surface;
a posterior surface; and
a shape formed into one of the anterior and posterior surfaces, said shape being defined by at least one undulating curvature along a meridian of the shape that produces different signs of longitudinal ray aberration within about 3 mm pupil diameter.

2. The lens according to claim 1 wherein said shape is a toric shape.

3. The lens according to claim 1 wherein said shape is a non-toric shape.

4. The lens according to claim 1 further wherein said shape is a tonic shape of two undulating curvatures along two meridians each producing different sips of longitudinal ray aberration with about 3 mm pupil diameter.

5. The lens according to claim 1 wherein a region of the same sign of the longitudinal ray aberration is at least 10% of the area within the 3 mm pupil diameter.

6. The lens according to claim 1 wherein the anterior and posterior surfaces are disposed on a contact lens.

7. The lens according to claim 1 wherein the anterior and posterior surfaces are disposed on an interocular lens.

8. The lens according to claim 1 wherein the anterior and posterior surfaces are disposed on a single optical element.

9. The lens according to claim 1 wherein the anterior and posterior surfaces are disposed on a multiple optical element.

10. The lens according to claim 1 wherein the anterior and posterior surfaces are disposed on a multifocal lens.

11. An ophthalmic toric lens for reducing sensitivity to meridianal misalignment, the lens comprising:
an anterior surface;
a posterior surface; and
a shape formed into one of the anterior and posterior surfaces, said toric shape being defined by at least one undulating curvature along a meridian of the shape that produces different signs of longitudinal ray aberration within about 3 mm pupil diameter.

12. The lens according to claim 11 wherein said shape is a toric shape.

13. The lens according to claims 11 wherein said shape is a non-toric shape.

14. The lens according to claim 11 wherein a region of different longitudinal ray aberration signs is at least 10% of the area within the 3 mm pupil diameter.

15. The lens according to claim 11 wherein the anterior and posterior surfaces are disposed on a contact lens.

16. The lens according to claim 11 wherein the anterior and posterior surfaces are disposed on an interocular lens.

17. The lens according to claim 11 wherein the anterior and posterior surfaces are disposed on a single optical element.

18. The lens according to claim 11 wherein the anterior and posterior surfaces are disposed on a multiple optical element.

19. The lens according to claim 11 wherein the anterior and posterior surfaces are disposed on a multifocal lens.
